(19) United States
(12) Patent Application Publication
(10) Pub. No.: US 2021/0065891 A1
(43) Pub. Date: Mar. 4, 2021

(54) Privacy-Preserving Activity Monitoring Systems And Methods

(71) Applicant: DawnLight Technologies Inc., Palo Alto, CA (US)

(72) Inventors: Jia Li, Palo Alto, CA (US); Ning Zhang, Mountain View, CA (US); Shuai Zheng, Campbell, CA (US); Jordan Hill Hurwitz, Burlingame, CA (US); Ziyu Zhang, Mountain View, CA (US); Mohammadhadi Kiapour, San Francisco, CA (US)

(21) Appl. No.: 16/552,846

(22) Filed: Aug. 27, 2019

(51) Int. Cl. G16H 40/67; G08B 21/02; G06N 3/08; G06N 3/04; A61B 5/11; A61B 5/00

(52) U.S. Cl. CPC G16H 40/67; G08B 21/02; G06N 3/08; G06N 3/049; A61B 5/1113; A61B 5/1117; A61B 5/746; A61B 5/1126; A61B 5/7264; A61B 5/1118

(57) ABSTRACT

Privacy-preserving activity monitoring systems and methods are described. In one embodiment, a plurality of sensors is configured for contact-free monitoring of at least one user state. A signal processing module communicatively coupled to the sensors is configured to receive data from the sensors. A first sensor is configured to generate a first set of quantitative data associated with a first user state. A second sensor is configured to generate a second set of quantitative data associated with a second user state. A third sensor is configured to generate a third set of quantitative data associated with a third user state. The signal processing module is configured to process the three sets of quantitative data using a machine learning module, and identify a user activity and detect a condition associated with the user, where no user-identifying information is communicated more than 100 meters to or from the signal processing module.

### BACKGROUND

### 1. Technical Field

The present disclosure relates to systems and methods that perform non-contact monitoring of one or more activities performed by an individual, using different sensing modalities and associated signal processing techniques that include machine learning.

### 2. Background Art

Currently, methods employed to monitor one or more activities (such as sitting, standing, sleeping, and so on) associated with a patient involve sensors attached to a patient's body, or methods that are potentially invasive to the patient's privacy. For example, using one or more cameras to monitor a patient's daily activity is associated with a potential privacy invasion, especially if data related to the monitoring is transmitted over a public network to a remote location. There exists a need for a non-contact (i.e., contact-free) and privacy-preserving method of monitoring one or more daily activities associated with a patient.

### SUMMARY

Embodiments of apparatuses configured to perform a contact-free monitoring of one or more user activities may include: a plurality of sensors configured to perform contact-free monitoring of at least one user state; and a signal processing module communicatively coupled with the plurality of sensors; wherein the signal processing module is configured to receive data from the plurality of sensors; wherein a first sensor of the plurality of sensors is configured to generate a first set of quantitative data associated with a first user state; wherein a second sensor of the plurality of sensors is configured to generate a second set of quantitative data associated with a second user state; wherein a third sensor of the plurality of sensors is configured to generate a third set of quantitative data associated with a third user state; wherein the signal processing module is configured to process the first set of quantitative data, the second set of quantitative data, and the third set of quantitative data using a machine learning module; wherein the signal processing module is configured to, responsive to the processing, identify a user activity and detect a condition associated with the user; and wherein no user-identifying information of the first through third sets of quantitative data and no user-identifying information of the processed data is communicated more than 100 meters from or to the signal processing module.

Embodiments of apparatuses configured to perform contact-free monitoring of one or more user activities may include one or all or any of the following:

The user activity may be any of sitting, standing, walking, sleeping, eating, undressing, dressing, washing face, washing hands, brushing teeth, brushing hair, using a toilet, putting on dentures, removing dentures, and/or laying down.

The condition may be any of a fall, a health condition, and/or a triage severity.

The signal processing module may be configured to generate an alarm in response to detecting a condition that is detrimental to the user.

The signal processing module and the plurality of sensors may be configured in a hub architecture; wherein the plurality of sensors are removably coupled with the signal processing module.

The signal processing module may include any of a GPU, an FPGA, and/or an AI computing chip.

The plurality of sensors may include any of a depth sensor, an RGB sensor, a thermal sensor, a radar sensor, and/or a motion sensor.

The signal processing module may characterize the user activity using a convolutional neural network.

The convolutional neural network may include a temporal shift module.

The signal processing module may be implemented using an edge device.

Embodiments of methods for performing contact-free monitoring of one or more user activities may include: generating, using a first sensor of a plurality of sensors, a first set of quantitative data associated with a first user state of a user, wherein the first sensor does not contact the user; generating, using a second sensor of the plurality of sensors, a second set of quantitative data associated with a second user state, wherein the second sensor does not contact the user; generating, using a third sensor of the plurality of sensors, a third set of quantitative data associated with a third user state, wherein the third sensor does not contact the user; processing, using a signal processing module and using a machine learning module, the first set of quantitative data, the second set of quantitative data, and the third set of quantitative data, wherein the signal processing module is communicatively coupled with the plurality of sensors; identifying, responsive to the processing, using the signal processing module, one or more user activities; and detecting, responsive to the processing, using the signal processing module, a condition associated with the user, wherein the plurality of sensors and the signal processing module are located at a healthcare campus, and wherein no user-identifying information of the first through third sets of quantitative data and no user-identifying information of the processed data is communicated offsite of the healthcare campus.

Embodiments of methods for performing contact-free monitoring of one or more user activities may include one or more or all of the following:

The user activity may be any of sitting, standing, walking, sleeping, eating, undressing, dressing, washing face, washing hands, brushing teeth, brushing hair, using a toilet, putting on dentures, removing dentures, and/or laying down.

The condition may be any of a fall, a health condition, and/or a triage severity.

The signal processing module may be configured to generate an alarm in response to detecting a condition that is detrimental to the user.

The signal processing module and the plurality of sensors may be configured in a hub architecture; wherein the plurality of sensors are removably coupled with the signal processing module.

The signal processing module may include any of a GPU, an FPGA, and/or an AI computing chip.

The plurality of sensors may include a thermal sensor, a radar sensor, and either or both of a depth sensor and an RGB sensor.

The user activity may be characterized using a neural network associated with the signal processing module.

The convolutional neural network may include a temporal shift module.

The signal processing module may be implemented using an edge device.

### BRIEF DESCRIPTION OF THE DRAWINGS

Non-limiting and non-exhaustive embodiments of the present disclosure are described with reference to the following figures, wherein like reference numerals refer to like parts throughout the various figures unless otherwise specified.

FIG. 1 is a block diagram depicting an embodiment of a remote health monitoring system implementation.

FIG. 2 is a block diagram depicting an embodiment of a signal processing module that is configured to implement certain functions of a remote health monitoring system.

FIG. 3 is a block diagram depicting an embodiment of an activity identification module.

FIG. 4 is a schematic diagram depicting a heatmap.

FIG. 5 is a block diagram depicting an embodiment of a system architecture of a remote health monitoring system.

FIG. 6 is a flow diagram depicting an embodiment of a method to detect a condition associated with a user.

FIG. 7 is a schematic diagram depicting a processing flow of multiple heatmaps using neural networks.

FIG. 8 is a schematic diagram depicting an embodiment of a temporal shift module.

FIG. 9 is a block diagram depicting an embodiment of a remote health monitoring system with privacy-preserving features.

FIG. 10 is a block diagram depicting an embodiment of a system architecture of a remote health monitoring system.

### DETAILED DESCRIPTION

In the following description, reference is made to the accompanying drawings that form a part thereof, and in which is shown by way of illustration specific exemplary embodiments in which the disclosure may be practiced. These embodiments are described in sufficient detail to enable those skilled in the art to practice the concepts disclosed herein, and it is to be understood that modifications to the various disclosed embodiments may be made, and other embodiments may be utilized, without departing from the scope of the present disclosure. The following detailed description is, therefore, not to be taken in a limiting sense.

Reference throughout this specification to “one embodiment,” “an embodiment,” “one example,” or “an example” means that a particular feature, structure, or characteristic described in connection with the embodiment or example is included in at least one embodiment of the present disclosure. Thus, appearances of the phrases “in one embodiment,” “in an embodiment,” “one example,” or “an example” in various places throughout this specification do not necessarily all refer to the same embodiment or example. Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

Embodiments in accordance with the present disclosure may be embodied as an apparatus, method, or computer program product. Accordingly, the present disclosure may take the form of an entirely hardware-comprised embodiment, an entirely software-comprised embodiment (including firmware, resident software, micro-code, etc.), or an embodiment combining software and hardware aspects that may all generally be referred to herein as a “circuit,” “module,” or “system.” Furthermore, embodiments of the present disclosure may take the form of a computer program product embodied in any tangible medium of expression having computer-usable program code embodied in the medium.

Any combination of one or more computer-usable or computer-readable media may be utilized. For example, a computer-readable medium may include one or more of a portable computer diskette, a hard disk, a random access memory (RAM) device, a read-only memory (ROM) device, an erasable programmable read-only memory (EPROM or Flash memory) device, a portable compact disc read-only memory (CDROM), an optical storage device, a magnetic storage device, and any other storage medium now known or hereafter discovered. Computer program code for carrying out operations of the present disclosure may be written in any combination of one or more programming languages. Such code may be compiled from source code to computer-readable assembly language or machine code suitable for the device or computer on which the code will be executed.

Embodiments may also be implemented in cloud computing environments. In this description and the following claims, “cloud computing” may be defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction and then scaled accordingly. A cloud model can be composed of various characteristics (e.g., on-demand self-service, broad network access, resource pooling, rapid elasticity, and measured service), service models (e.g., Software as a Service (“SaaS”), Platform as a Service (“PaaS”), and Infrastructure as a Service (“IaaS”)), and deployment models (e.g., private cloud, community cloud, public cloud, and hybrid cloud).

The flow diagrams and block diagrams in the attached figures illustrate the architecture, functionality, and operation of possible implementations of systems, methods, and computer program products according to various embodiments of the present disclosure. In this regard, each block in the flow diagrams and/or block diagrams may represent a module, segment, or portion of code, which includes one or more executable instructions for implementing the specified logical function(s). It will also be noted that each block of the block diagrams and/or flow diagrams, and combinations of blocks in the block diagrams and/or flow diagrams, may be implemented by special purpose hardware-based systems that perform the specified functions or acts, or combinations of special purpose hardware and computer instructions. These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flow diagram and/or block diagram block or blocks.

The systems and methods described herein relate to a remote health monitoring system that is configured to monitor and identify one or more activities associated with a patient or a user, and detect a condition associated with the user. In some embodiments, the one or more activities include activities of daily life such as sitting, standing, walking, sleeping, eating, and laying down. Some embodiments of the remote health monitoring system use multiple sensors with associated signal processing and machine learning to perform the identification and detection processes, as described herein.

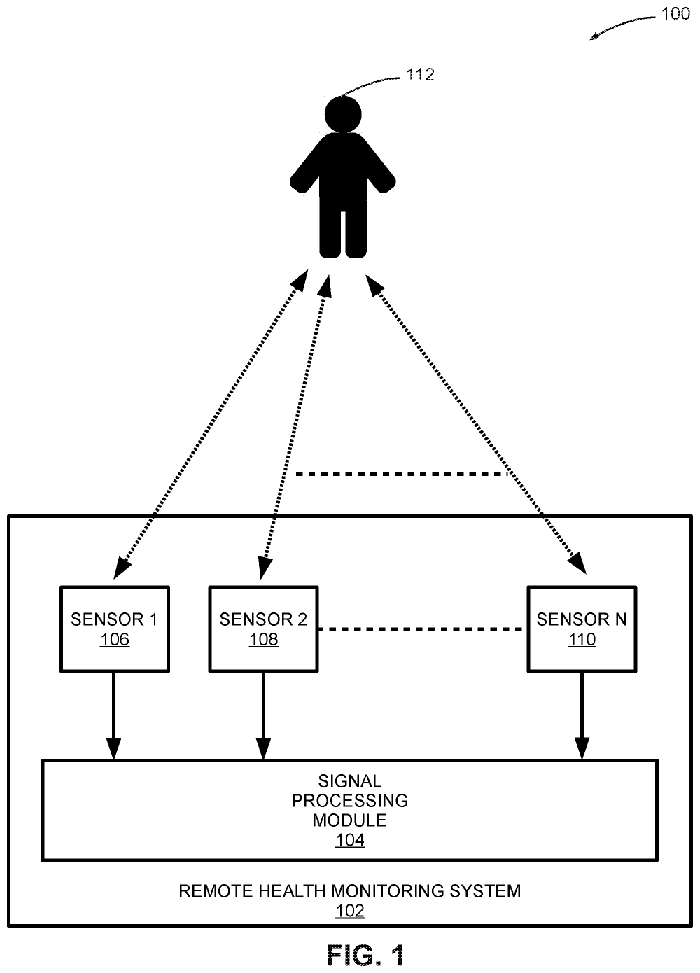

FIG. 1 is a block diagram depicting an embodiment of a remote health monitoring system implementation 100. In some embodiments, remote health monitoring implementation 100 includes a remote health monitoring system 102 that is configured to identify an activity and detect a condition associated with a user 112. In particular embodiments, remote health monitoring system 102 is configured to identify the activity and detect the condition, using a sensor 1 106, a sensor 2 108, through a sensor N 110 included in remote health monitoring system 102. In some embodiments, remote health monitoring system 102 includes a signal processing module 104 that is communicatively coupled to each of sensor 1 106 through sensor N 110, where signal processing module 104 is configured to receive data generated by each of sensor 1 106, through sensor N 110.

In some embodiments, each of sensor 1 106 through sensor N 110 is configured to remotely measure and generate data associated with a bodily function of user 112, in a contact-free manner. For example, sensor 1 106 may be configured to generate a first set of quantitative data associated with a measurement of a user speed and a user position; sensor 2 108 may be configured to generate a second set of quantitative data associated with a measurement of a user action; and sensor N 110 may be configured to generate a third set of quantitative data associated with a measurement of a user movement.

In some embodiments, the user speed and the user position may include a speed of motion (e.g., walking) of user 112, and a position of user 112 in an environment such as a room. In some embodiments, the first set of quantitative data associated with the user speed and the user position may be stored in memory to enable a temporal tracking ability associated with signal processing module 104.

In some embodiments, the user action may include any combination of eating, sitting, standing, walking, lying in bed, reclining in a reading position, taking off clothes (undressing), wearing clothes (dressing), brushing teeth, brushing hair, using a toilet, washing face, washing hands, putting on dentures, removing dentures, and so on. The user action, as defined herein, may or may not include movement. For example, the user may be sitting in a chair not moving, or standing still, or lying in bed without moving, and these are still considered “user actions” as that phrase is used herein. In particular embodiments, the user movement may be any combination of walking, getting out of a chair, a process of laying down in bed, eating, and so on. Accordingly, there may be some overlap between user actions and user movement, inasmuch as some user actions involve movement. Collectively, the user speed and the user position, the user action, and the user movement are used to characterize an activity of daily life (ADL), also referred to herein as an “activity,” a “daily activity,” or a “user activity.” Non-exhaustive examples of activities include sitting, walking, lying down, sitting down into a chair, getting out of the chair, eating, sleeping, getting out of bed, standing, falling, reading, watching TV, using a cell phone, and so on. Accordingly, there may be some overlap between the user actions, user movements, and the activity of daily life.

In some embodiments, signal processing module 104 is configured to process the first set of quantitative data, the second set of quantitative data, and the third set of quantitative data to identify a user activity and detection of a condition associated with user 112, where the condition can be any of a fall, a health condition, and a triage severity. In particular embodiments, signal processing module 104 may use a machine learning algorithm to process at least one of the sets of quantitative data, as described herein.

In some embodiments, data processed by signal processing module 104 may include current (or substantially real-time) data that is generated by sensor 1 106 through sensor N 110 at a current time instant. In other embodiments, data processed by signal processing module 104 may be historical data generated by sensor 1 106 through sensor N 110 at one or more earlier time instants. In still other embodiments, data processed by signal processing module 104 may be a combination of substantially real-time data and historical data.

In some embodiments, each of sensor 1 106 through sensor N 110 is a contact-free (or contactless, or non-contact) sensor, which implies that each of sensor 1 106 through sensor N 110 is configured to function with no physical contact or minimal physical contact with user 112. For example, sensor 1 106 may be a radar that is configured to remotely perform ranging and detection functions associated with a bodily function such as heartbeat or respiration; sensor 2 108 may be a visual sensor that is configured to remotely sense user actions; and sensor N 110 may be a motion sensor that is configured to remotely sense a motion associated with user 112. In some embodiments, the radar is a millimeter wave radar, the visual sensor is a depth sensor or a red-green-blue (RGB) sensor, and the motion sensor is an infrared (IR) sensor. Operational details of example sensors that may be included in a group comprising sensor 1 106 through sensor N 110 are provided herein. Additionally, any of the sensors could be a combination of sensor types, for example the visual sensor could include a depth sensor and an RGB sensor.

Using non-contact sensing for implementing remote health monitoring system 102 provides several advantages. Non-contact sensors make an implementation of remote health monitoring system 102 non-intrusive and easy to set up in, for example, a home environment for long term continuous monitoring. Also, from a perspective of compliance with health standards, remote health monitoring system 102 requires minimal to no efforts on behalf of a patient (i.e., user 112) to install and operate the system; hence, such an embodiment of remote health monitoring system 102 would not violate any compliance regulations.

One example operation of remote health monitoring system 102 is based on the following steps:
Analyze performance of activities of daily living to detect acute changes and increasing care need associated with user 112.
Detect falls, triage severity and predict such events in advance.

A benefit of this approach is that it provides families peace of mind that caretakers are taking care of their loved ones as needed. Some embodiments of remote health monitoring system 102 include signal processing module 104 receiving data from sensor 1 106 through sensor N 110 and processing this data locally (i.e., where signal processing module 104 is located in a vicinity of user 112). In particular embodiments, a maximum distance between signal processing module 104 and user 112, or between signal processing module 104 and any of sensor 1 106 through sensor N 110, is 100 meters or less. In other implementations there may be greater or shorter distances between the elements. Furthermore, in implementations all data processing by signal processing module 104 is performed on signal processing module 104, without signal processing module 104 sending any such data over, for example, a public network, to a remote computing device such as a remote server or a cloud computing device. This aspect of remote health monitoring system 102 ensures that no sensitive user-related data is transmitted over a public network. In some embodiments, signal processing module 104 is implemented on an edge device. Essentially, privacy-preserving signals (i.e., data related to user 112 as generated by sensor 1 106 through sensor N 110) are locally processed on signal processing module 104, without sending this data to the cloud. Potential applications of remote health monitoring system include ADL recognition, chronic disease management, and remote healthcare.

Advantages of remote health monitoring implementation 100 include:
Privacy protection: Provide different levels of privacy solutions for different scenarios.
Contactless monitoring: Using contact-free monitoring relieves a patient from an inconvenience associated with wearing one or more wearable pieces of portable equipment (e.g., a mask).
24/7 monitoring: Enables patients and seniors to receive round-the-clock care monitoring, even when caregivers are not in a vicinity of a patient or user.
Increased effectiveness: Increased effectiveness of in-home visits with deeper understanding in-between visits.
One-to-many monitoring: One device can monitor multiple targets (users or patients) in a certain space or environment.
Contact-free monitoring and local processing allow high compliance with existing health standards.
Enabling a sensor fusion approach coupled with machine learning signal processing techniques provides a high-precision, low-cost solution.

In some embodiments, remote health monitoring system 102 includes signal processing module 104, and sensor 1 106 through sensor N 110, integrated into a single enclosure, casing or housing. In other embodiments, signal processing module 104 and sensor 1 106 through sensor N 110 can be configured such that signal processing module 104 is a hub, and each of sensor 1 106 through sensor N 110 are satellites, as discussed herein.

In some embodiments, sensor 1 106 through sensor N 110 can be any combination of a depth sensor, a thermal sensor, a radar sensor, a motion sensor, and any other privacy-preserving sensors. In other implementations non-privacy preserving sensors may be used, but the system may remove all private information so that privacy is maintained. In some embodiments, signal processing module 104 may be enabled to perform AI computation using any combination of an artificial intelligence (AI) computing chip, a graphics processing unit (GPU), a central processing unit (CPU), a digital signal processor (DSP), a microcontroller, a field-programmable gate array (FPGA), a complex programmable logic device (CPLD), or any other kind of computing device. In particular embodiments, all communication and coupling links (e.g., coupling between each of sensor 1 106 through sensor N 110 and signal processing module 104) can be implemented using any combination of wireless and wireless communication links such as WiFi, Bluetooth, 4G, 5G, serial peripheral interface (SPI), Ethernet, a parallel port, a serial port, a universal serial bus (USB) interface, and so on.

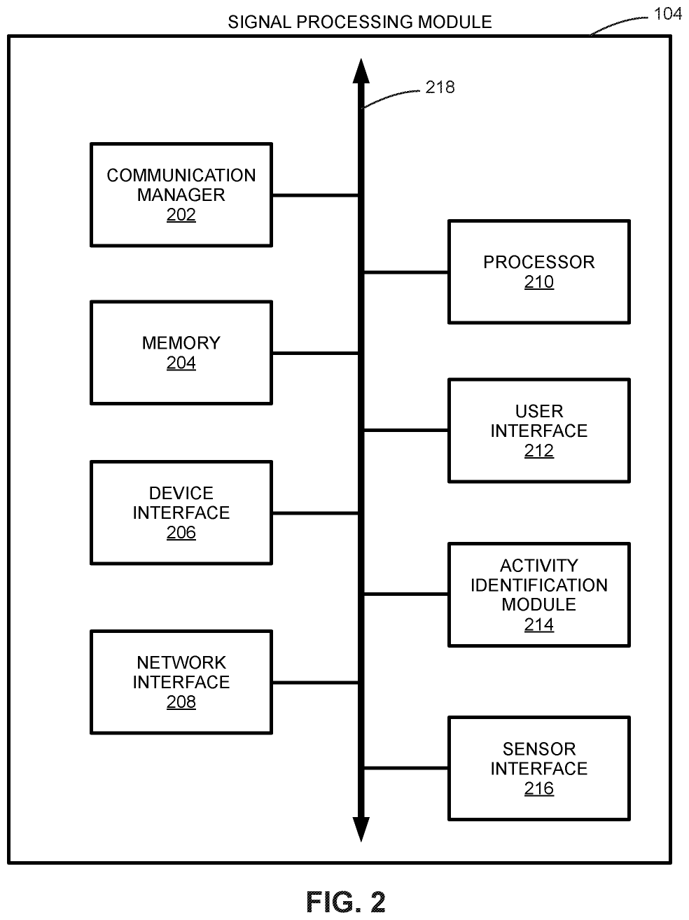

FIG. 2 is a block diagram depicting an embodiment of a signal processing module 104 that is configured to implement certain functions of remote health monitoring system 102. In some embodiments, signal processing module 104 includes a communication manager 202, where communication manager 202 is configured to manage communication protocols and associated communication with external peripheral devices as well as communication within other components in signal processing module 104. For example, communication manager 202 may be responsible for generating and maintaining associated interfaces between signal processing module 104 and each of sensor 1 106 through sensor N 110. Communication manager 202 may also be responsible for managing communication between the different components within signal processing module 104.

Some embodiments of signal processing module 104 include a memory 204 that may include both short-term memory and long-term memory. Memory 204 may be used to store, for example, substantially real-time and historical quantitative data sets generated by sensor 1 106 through sensor N 110. Memory 204 may be comprised of any combination of hard disk drives, flash memory, random access memory, read-only memory, solid state drives, and other memory components.

In some embodiments, signal processing module 104 includes a device interface 206 that is configured to interface signal processing module 104 with one or more external devices such as an external hard drive, an end user computing device (e.g., a laptop computer or a desktop computer), and so on. Device interface 206 generates any necessary hardware communication protocols associated with one or more communication protocols such as a serial peripheral interface (SPI), a serial interface, a parallel interface, a USB interface, and so on.

A network interface 208 included in some embodiments of signal processing module 104 includes any combination of components that enable wired and wireless networking to be implemented. Network interface 208 may include an Ethernet interface, a WiFi interface, and so on. In some embodiments, network interface 208 allows remote health monitoring system 102 to send and receive data over a local network or a public network.

Signal processing module 104 also includes a processor 210 configured to perform functions that may include generalized processing functions, arithmetic functions, and so on. Signal processing module 104 is configured to process one or more sets of quantitative data generated by sensor 1 106 through sensor N 110. Any artificial intelligence algorithms or machine learning algorithms (e.g., neural networks) associated with remote health monitoring system 102 may be implemented using processor 210.

In some embodiments, signal processing module 104 may also include a user interface 212, where user interface 212 may be configured to receive commands from user 112 (or another user, such as a health care worker, family member or friend of the user 112, etc.), or display information to user 112 (or another user). User interface 212 enables a user to interact with remote health monitoring system 102. In some embodiments, user interface 212 includes a display device to output data to a user; one or more input devices such as a keyboard, a mouse, a touchscreen, one or more push buttons, one or more switches; and other output devices such as buzzers, loudspeakers, alarms, LED lamps, and so on.

Some embodiments of signal processing module 104 include an activity identification module 214 that is configured to process a plurality of sets of quantitative data generated by sensor 1 106 through sensor N 110 in conjunction with processor 210, and identify an activity and detect a condition associated with user 112. In some embodiments, activity identification module 214 processes the plurality of sets of quantitative data using one or more machine learning algorithms such as neural networks, linear regression, a support vector machine, and so on. Details about activity identification module 214 are presented herein.

In some embodiments, signal processing module 104 includes a sensor interface 216 that is configured to implement necessary communication protocols that allow signal processing module 104 to receive data from sensor 1 106, through sensor N 110.

A data bus 218 included in some embodiments of signal processing module 104 is configured to communicatively couple the components associated with signal processing module 104 as described above.

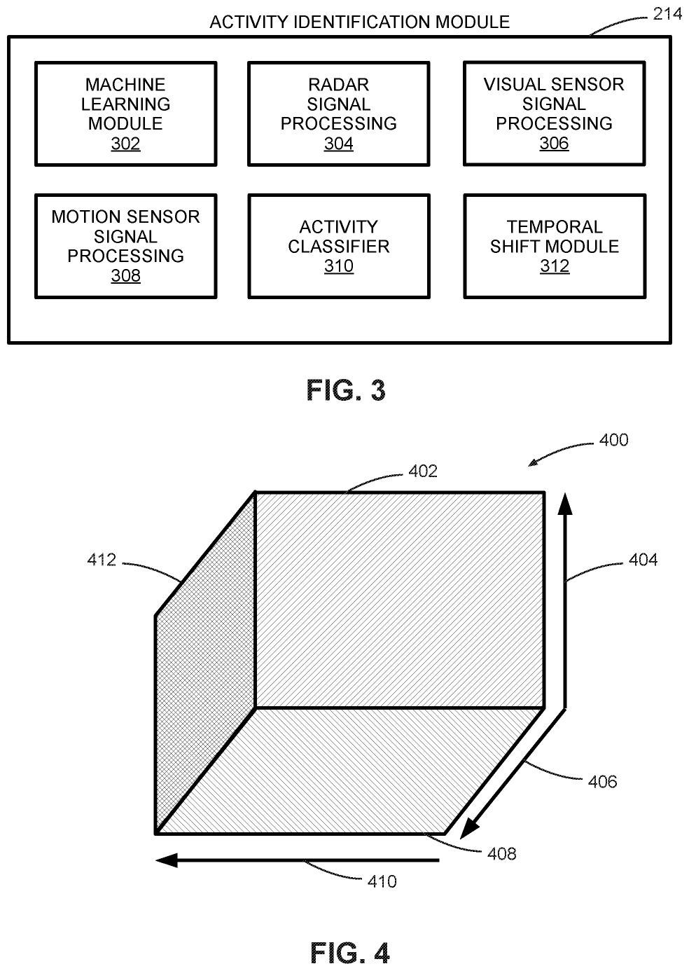

FIG. 3 is a block diagram depicting an embodiment of activity identification module 214. In some embodiments, activity identification module 214 includes a machine learning module 302 that is configured to implement one or more machine learning algorithms that enable remote health monitoring system 102 to intelligently identify an activity and detect a condition associated with user 112. In some embodiments, machine learning module 302 is used to implement one or more machine learning structures such as a neural network, a linear regression, a support vector machine (SVM), or any other machine learning algorithm. In implementations, for large sets of quantitative data a neural network is a preferred algorithm in machine learning module 302.

In some embodiments, activity identification module 214 includes a radar signal processing 304 that is configured to process a set of quantitative data generated by a radar sensor included in sensor 1 106 through sensor N 110. Activity identification module 214 also includes a visual sensor signal processing 306 that is configured to process a set of quantitative data generated by a visual sensor included in sensor 1 106 through sensor N 110. Activity identification module 214 also includes a motion sensor signal processing 308 that is configured to process a set of quantitative data generated by a motion sensor included in sensor 1 106 through sensor N 110.

In some embodiments, activity identification module 214 includes an activity classifier 310 that is configured to classify one or more activities associated with user 112, responsive to activity identification module 214 processing one or more sets of quantitative data generated by sensor 1 106 through sensor N 110.

In some embodiments, activity identification module 214 includes a temporal shift module 312 that is configured to process one or more video frames generated by a visual sensor and generate an output that is used to predict an action by user 112. Details about temporal shift module 312 are provided herein.

FIG. 4 is a schematic diagram depicting a heatmap 400. In some embodiments, heatmap 400 is generated responsive to signal processing module 104 processing a set of quantitative data generated by a radar. Details about the radar used in remote health monitoring system 102 are described herein. In particular embodiments, the set of quantitative data is processed by radar signal processing 304, where the radar is configured to generate quantitative data associated with radio frequency (RF) signal reflections. In some embodiments, the radar is a millimeter wave frequency-modulated continuous wave radar (FMCW).

In some embodiments, heatmap 400 is generated based on a view 412 associated with the radar. View 412 is a representation of a view of an environment associated with user 112, where user 112 is included in a field of view of the radar. Responsive to processing RF reflection data associated with view 412, radar signal processing 304 generates a horizontal-depth heatmap 408 and a vertical-depth heatmap 402, where each of horizontal-depth heatmap 408 and vertical-depth heatmap 402 are referenced to a vertical axis 404, a horizontal axis 406, and a depth axis 410. In some embodiments, heatmap 400 is used as a basis for generating one or more sets of quantitative data associated with a heartbeat and a respiration of user 112.

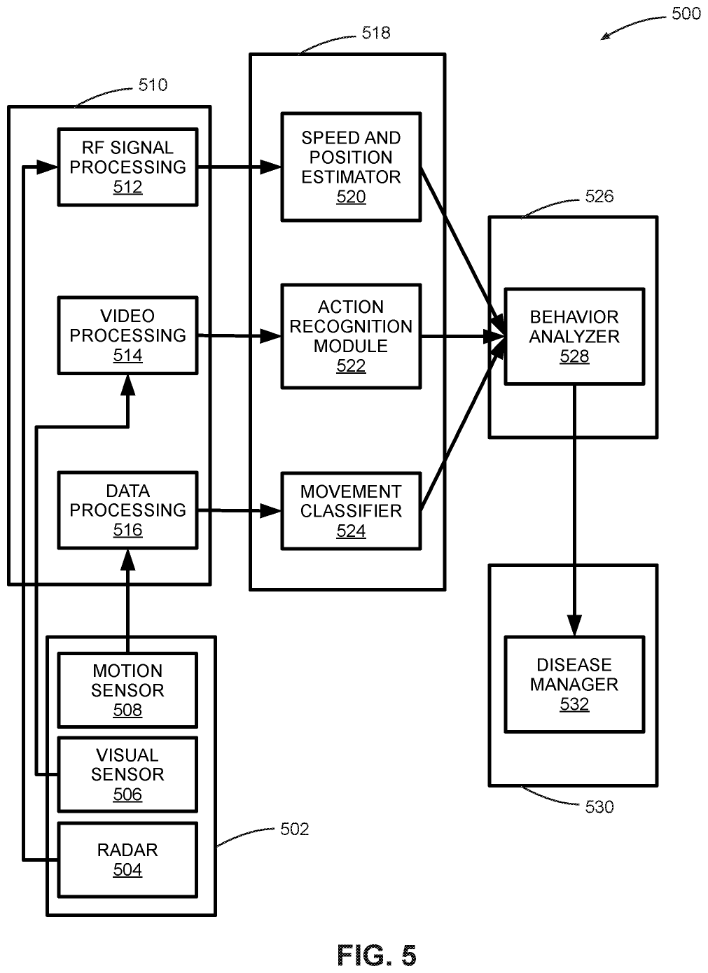

FIG. 5 is a block diagram depicting an embodiment of a system architecture 500 of a remote health monitoring system. In some embodiments, system architecture 500 includes a sensor layer 502. Sensor layer 502 includes a plurality of sensors configured to generate one or more sets of quantitative data associated with measuring one or more bodily functions associated with user 112. In some embodiments, sensor layer 502 includes sensor 1 106 through sensor N 110. In particular embodiments, sensor layer 502 includes a radar 504, a visual sensor 506, and a motion sensor 508.

In some embodiments, radar 504 is a millimeter wave frequency-modulated continuous wave radar that is designed for indoor use. Visual sensor 506 is configured to generate visual data associated with user 112. In some embodiments, visual sensor may include a depth sensor and/or an RGB sensor. Motion sensor 508 is configured to generate data associated with a motion of user 112. In some implementations the motion sensor only detects a scene change without reference to whether the scene change is due to movement of a person, or a light switching on/off, and so forth. In implementations the motion detector may repeatedly check for scene changes and, if the motion detector does not detect a scene change, the other sensors may remain inactive, whereas when the motion detector detects a scene change the other sensors begin data collection. In other implementations the other detectors may remain active, and gather data, regardless of whether the motion sensor detects a scene change.

In some embodiments, system architecture 500 includes a detection layer 510 that is configured to receive and process one or more sets of quantitative data generated by sensor layer 502. Detection layer 510 is configured to receive a set of quantitative data (also referred to herein as “sensor data”) from sensor layer 502. Detection layer 510 processes this sensor data to extract signals associated with a user activity from the sensor data. In particular embodiments, detection layer 510 includes an RF signal processing 512 that is configured to receive sensor data from radar 504, a video processing 514 that is configured to received sensor data from visual sensor 506, and a data processing 516 that is configured to receive sensor data from motion sensor 508.

In some embodiments, radar 504 is a millimeter wave frequency-modulated continuous wave radar. Radar 504 is capable of capturing fine motions of user 112 that include breathing and a heartbeat, as well as larger-scale motions such as walking, sitting down in a chair, and so on. In particular embodiments, sensor data generated by radar 504 is processed by RF signal processing 512 to generate a heatmap such as heatmap 400.

In some embodiments, visual sensor 506 includes a depth sensor and/or an RGB sensor. Visual sensor 506 is configured to capture visual data associated with user 112. In some embodiments, this visual data includes data associated with user actions performed by user 112. These user actions may include walking, lying down into a bed, maintaining a lying position, sitting down into a chair, maintaining a sitting position, getting out of the chair, eating, sleeping, standing, taking off clothes (undressing), wearing clothes (dressing), brushing teeth, brushing hair, using a toilet, washing face, washing hands, putting on dentures, removing dentures, and so on. In particular embodiments, this visual data generated by visual sensor 506, output as sensor data from visual sensor 506, is processed by video processing 514 to extract ADL features associated with daily activities described above, and features such as a sleep quality, a meal quality, a daily calorie burn rate estimation, a frequency of coughs, a visual sign of breathing difficulty, and so on. In some embodiments, video processing 514 uses machine learning algorithms such as a combination of a neural network, a linear regression, a support vector machine, and other machine learning algorithms.

In some embodiments, a sensing capability associated with visual sensor 506 may be complemented by one or more thermal sensors included in sensor layer 502. (A thermal sensor is not depicted in FIG. 5.) The thermal sensor may be useful for providing data even when other sensors, such as a depth sensor and/or an RGB sensor, cannot detect the user position and/or movement because of the user being occluded by something (for example the entire user's body may be occluded by blankets during sleep, but the thermal sensor may still detect the user's position and/or movement due to the warm body of the user). An output generated by the thermal sensors is received and processed by video processing 514.

Some embodiments of video processing 514 use a temporal spatial convolutional neural network, which takes a feature from a frame at a current time instant, and copies part of the feature to a next time frame. At each time frame, the temporal spatial convolutional neural network (also known as a “model”) will predict a type of activity, e.g. sitting, walking, falling, or no activity. Since an associated model generated by video processing 514 copies one or more portions of features from a current timestamp to a next timestamp, video processing 514 learns a temporal representation aggregated from a period of time to predict an associated activity. In some embodiments, this process is implemented using a temporal shift module as described herein.

In some embodiments, motion sensor 508 is configured to detect a motion associated with user 112. Motion sensor 508 is configured to generate quantitative data associated with this motion. This quantitative data is received by data processing 516 that is configured to process the data and extract features associated with the motion. In some embodiments, motion sensor 508 is an infrared sensor. In particular embodiments, the infrared sensor includes two slots that detect a substantially identical amount, or an identical amount, of infrared light when there is no user motion, and detect a positive differential change between the two slots when a warm body like a human or animal passes by. Data processing 516 receives this differential change and accordingly outputs a signal strength associated with any existing motion. In implementations data processing module 516 simply outputs a binary output indicating either “motion” or “no motion.”

In some embodiments, one or more outputs generated by detection layer 510 are received by a signal layer 518. Signal layer 518 is configured to quantify data generated by detection layer 510. In particular embodiments, signal layer 518 generates one or more time series in response to the quantification. Specifically, signal layer 518 includes a speed and motion estimator 520 that is configured to receive an output generated by RF signal processing 512; an action recognition module 522 that is configured to receive an output generated by video processing 514; and a movement classifier 524 that is configured to receive an output generated by data processing 516.

In some embodiments, speed and motion estimator 520 is configured to process data received from RF signal processing 512 to generate an estimate of a speed and position associated with user 112. For example, a certain speed and position may be associated with user 112 engaging in daily activities such walking, sitting down or getting out of a chair, and so on. On the other hand, a sudden vertical motion profile with a corresponding relatively large vertical velocity may indicate that user 112 may have fallen.

In some embodiments, action recognition module 522 is configured to process data received from video processing 514 to determine an action associated with user 112. As described earlier, examples of actions include walking, eating, laying down, sitting, and so on. In particular embodiments, action recognition module 522 processes data received from video processing 514 using a two-dimensional convolutional neural network (2D CNN) that includes a temporal shift module (TSM). In other embodiments, action recognition module 522 processes data received from video processing 514 using a three-dimensional convolutional neural network (3D CNN).

In some embodiments, data generated by visual sensor 506 is a set of video frames. Video processing 514 processes these video frames to extract user actions indicative of ADL features from the video frames, and then passes these video frames to action recognition module 522. In action recognition module 522, each video frame is fed into a 2D convolutional neural network. Using a 2D CNN independently for each frame does not capture any temporal information associated with the video frames. A TSM used in conjunction with a 2D CNN shifts parts of channels associated with a stream of video frames along an associated temporal dimension, which can use temporal information among neighboring video frames. This can enable temporal modeling in an efficient way.

In some embodiments, movement classifier 524 is configured to receive data from data processing 516 and classify a movement associated with user 112, where the movement is associated with dynamic body motions such as walking, getting in or out of bed, eating, and so on. This classification in implementations is performed by movement classifier 524 learning a linear classifier to make a binary determination, based on data from the motion sensor, of whether to output “motion” or “no motion” (or, in other words, a signal indicating motion or a signal indicating no motion). In other implementations the movement classifier may be more complex and also output types of motions, such as walking, washing hands, etc. In implementations the movement classifier may be excluded and the output from data processing 516 may be routed directly to the behavior analyzer, or the movement classifier may simply forward the output from data processing 516 without further analysis or modification, with the data being simply an indication of whether a scene change was detected or not (for example “motion” or “no motion” at a given time instance). In other implementations data processing module 516 and movement classifier 524 could be excluded, and motion sensor 508 could directly output a binary “motion” or “no motion” to behavior analyzer 528. In any case, if no scene change was detected, the behavior analyzer may determine that there are likely no user actions associated with that time instance.

In some embodiments, outputs generated by signal layer 518 are received by a model layer 526 that is configured to process these outputs using behavior analysis based on machine learning. Specifically, a behavior analyzer 528 is configured to receive an output generated by each of speed and position estimator 520, action recognition module 522, and movement classifier 524. Behavior analyzer 528 implements behavior analysis machine learning algorithms to analyze and determine a behavior associated with user 112, based on a speed and a position associated with user 112 (as determined by speed and position estimator 520), an action associated with user 112 (as determined by action recognition module 522), and a classification of a movement associated with user 112 (as determined by movement classifier 524).

In some embodiments, an output generated by model layer 526 is received by an application layer 530. Specifically, an output generated by behavior analyzer 528 is received by a disease manager 532 that is associated with application layer 530. Disease manager 532 is configured to enable remote health monitoring system 102 to perform chronic disease management associated with user 112. For example, disease manager 532 may determine that user 112 might have fallen. Or, disease manager 532 may determine that user 112 may be suffering from an attack of a chronic disease such as asthma, based on a movement of user 112 being sluggish on a particular day as compared to a recorded movement history of user 112 on a day when a health of user 112 is good. In another example, disease manager 532 may determine a progress of a disease such as stroke rehabilitation based on one or more movements associated with user 112.

In some embodiments, system architecture 500 is configured to fuse, or blend data from multiple sensors such as sensor 1 106 through sensor N 110 (shown as radar 504, visual sensor 506, and motion sensor 508 in FIG. 5), and identify a user activity and detect a condition associated with user 112. In some embodiments, outputs generated by sensor 1 106 through sensor N 110 are processed by remote health monitoring system 102 in real-time to provide real-time alerts associated with a health condition such as a fall, an asthma attack, or a triage severity, that is detrimental to user 112. These real-time alerts include alarms when remote health monitoring detects a condition that is detrimental to user 112. In other embodiments, remote health monitoring system 102 uses historical data and historical statistics associated with user 112 to determine one or more conditions associated with user 112. In still other embodiments, remote health monitoring system 102 is configured to use a combination of real-time data generated by sensor 1 106 through sensor N 110 along with historical data and historical statistics associated with user 112 to determine one or more conditions associated with user 112.

Using a sensor fusion approach allows for a greater confidence level in detecting and diagnosing a condition associated with user 112. Using a single sensor is prone to increasing a probability associated with incorrect predictions, especially when there is an occlusion, a blindspot, a long range or multiple people in a scene as viewed by the sensor. Using multiple sensors in combination, and combining data processing results from processing discrete sets of quantitative data generated by the various sensors, produces a more accurate prediction, as different sensing modalities complement each other in their capabilities.

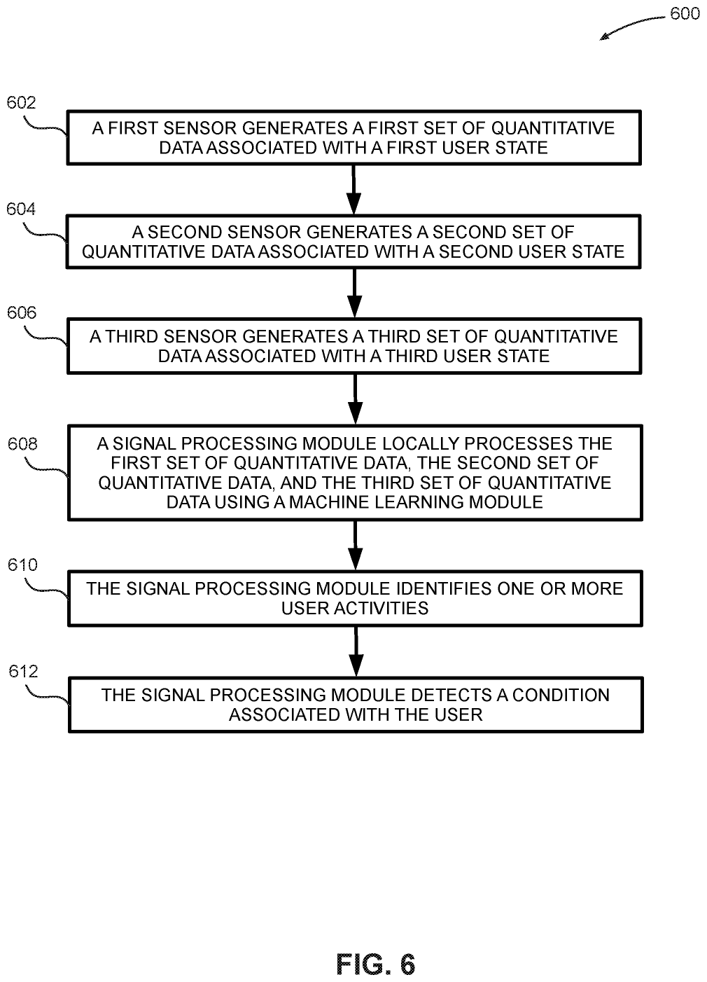

FIG. 6 is a flow diagram depicting an embodiment of a method 600 to detect a condition associated with a user. At 602, a first sensor generates a first set of quantitative data associated with a user speed and a user position. In some embodiments, the first sensor is radar 504, and the first set of quantitative data is associated with one or more RF signals received by radar 504, where the RF signals include position and speed information (e.g., Doppler shifts). At 604, a second sensor generates a second set of quantitative data associated with a user action. In some embodiments, the second sensor is visual sensor 506, the second set of quantitative data is associated with one or more visual signals generated by visual sensor 506, and the second set of quantitative data is associated with an action performed by user 112. At 606, a third sensor generates a third set of quantitative data associated with a user movement. In some embodiments, the third sensor is motion sensor 508, the third set of quantitative data is associated with one or more motion signals received by motion sensor 508, and the third set of quantitative data is associated with a movement performed by user 112. At 608, a signal processing module processes the first set of quantitative data, the second set of quantitative data, and the third set of quantitative data using a machine learning module. In some embodiments the signal processing module is signal processing module 104 that is configured to implement detection layer 510, signal layer 518, model layer 526, and application layer 530. At 610, the signal processing module identifies one or more user activities as described herein. Finally, at 612, the signal processing module detects a condition associated with the user. In some embodiments, the condition may be any combination of a fall, a health condition (e.g., asthma or COPD), or a triage severity. In implementations, however, any of the layers may have different, more, or fewer elements to diagnose different, or more, or fewer health conditions. In implementations one or more of the steps of method 600 may be performed in a different order than that presented.

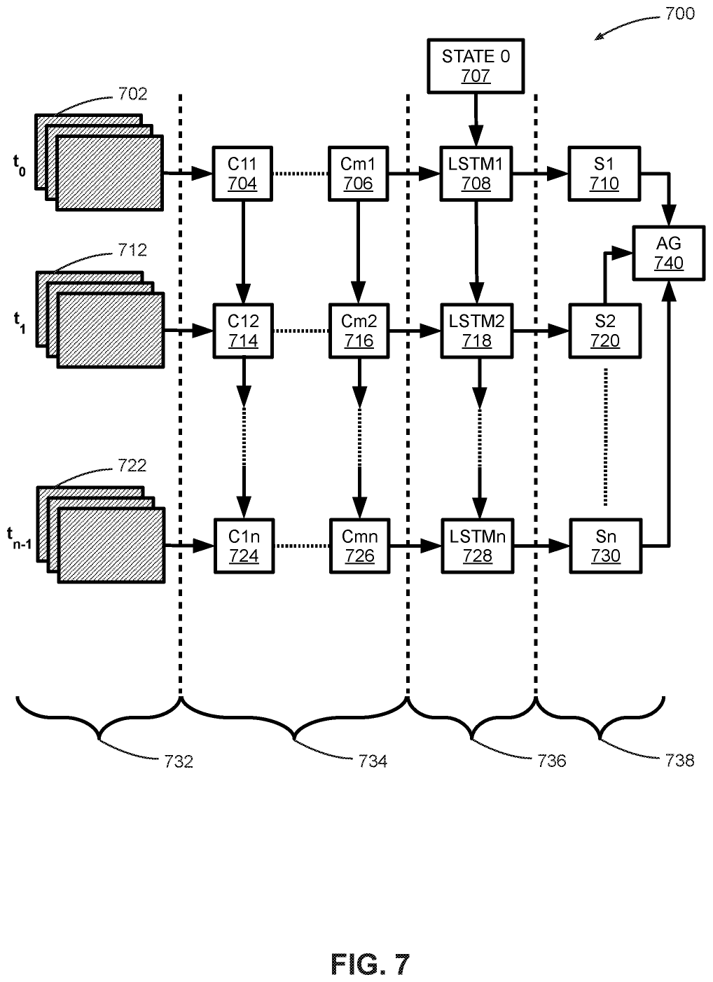

FIG. 7 is a schematic diagram depicting a processing flow 700 of multiple heatmaps using neural networks. In some embodiments, processing flow 700 is configured to function as a fall classifier that determines whether user 112 has had a fall. In some embodiments, processing flow 700 processes a temporal set of heatmaps 732 that includes a first set of heatmaps 702 at a time t₀, a second set of heatmaps 712 at a time t₁, through an n^{th} set of heatmaps 722 at a time tₙ₋₁. In implementations, receiving temporal set of heatmaps 732 comprises a preprocessing phase for processing flow 700.

In some embodiments, time t₀, time t₁ through time tₙ₋₁ are consecutive time steps, with a fixed-length sliding window (e.g., 5 seconds). Temporal set of heatmaps 732 is processed by a multi-layered convolutional neural network 734. Specifically, first set of heatmaps 702 is processed by a first convolutional layer C11 704 and so on, through an m^{th} convolutional layer Cm1 706; second set of heatmaps 712 is processed by a first convolutional layer C12 714 and so on, through an m^{th} convolutional layer Cm2 716; and so on through n^{th} set of heatmaps 722 being processed by a first convolutional layer C1n 724, through an m^{th} convolutional layer Cmn 726. In some embodiments, a convolutional layer with generalized indices Cij is configured to receive an input from a convolutional layer C(i−1)j for i>1, and a convolutional layer Cij is configured to receive an input from convolutional layer Ci(j−1) for j>1. For example, convolutional layer Cm2 716 is configured to receive an input from a convolutional layer C(m−1)2 (not shown in FIG. 7), and from convolutional layer Cm1 706. In some embodiments, an input received by convolutional layer Cij from convolutional layer Ci(j−1) comprises a temporal shift. For example, an input received by convolutional layer C12 714 from convolutional layer C11 704 comprises a temporal shift, and so on. In this sense, an ensemble of convolution layers associated with processing flow 700 includes a temporal shift module.

Collectively, first convolutional layer C11 704 through m^{th} convolutional layer Cm1 706, first convolutional layer C12 714, through m^{th} convolutional layer Cm2 716 and so on, through first convolutional layer C1n 724, through m^{th} convolutional layer Cmn 726 comprise multi-layered convolutional neural network 734 that is configured to extract salient features at each timestep, for each of the first set of heatmaps 702 through the n^{th} set of heatmaps 722.

In some embodiments, outputs generated by multi-layered convolutional neural network 734 are received by a recurrent neural network 736 that is comprised of a long short-term memory LSTM1 708, a long short-term memory LSTM2 718, through a long short-term memory LSTMn 728. In some embodiments, long short-term memory LSTM1 708 is configured to receive an output from m^{th} convolutional layer Cm1 706 and an initial system state 0 707, long short-term memory LSTM2 718 is configured to receive inputs from long short-term memory LSTM1 708 and m^{th} convolutional layer Cm2 716 and so on, through long short-term memory LSTMn 728 being configured to receive inputs from a long short-term memory LSTM(n−1) (not shown but implied in FIG. 7) and m^{th} convolutional layer Cmn 726. Recurrent neural network 736 is configured to capture complex spatio-temporal dynamics associated with temporal set of heatmaps 732 while taking into account the multiple discrete time steps t₀ through tₙ₋₁.

In some embodiments, an output generated by each of long short-term memory LSTM1 708, long short-term memory LSTM2 718, through long short-term memory LSTMn 728 is received by a softmax S1 710, a softmax S2 720, and so on through a softmax Sn 730, respectively. Collectively, softmax S1 710, softmax S2 720 through softmax Sn 730 comprise a classifier 738 that is configured to categorize an output generated by the corresponding recurrent neural network to determine, for example, whether user 112 has had a fall at a particular time instant in a range of t₀ through tₙ₋₁.

In some embodiments, an output of each of softmax S1 710 through softmax Sn 730 is received by an aggregator AG 740 that is configured to aggregate data received by aggregator AG 740 from softmax S1 710 through softmax Sn 730. Each of softmax S1 710 through softmax Sn 730 is configured to process data associated with a particular time instant. Aggregator AG 740 receives collective data associated with a time interval that is comprised of the instances of time associated with each of softmax S1 710 through softmax Sn 730. In some embodiments, aggregator AG 740 is configured to determine a final prediction associated with a user condition, responsive to processing data from the time interval.

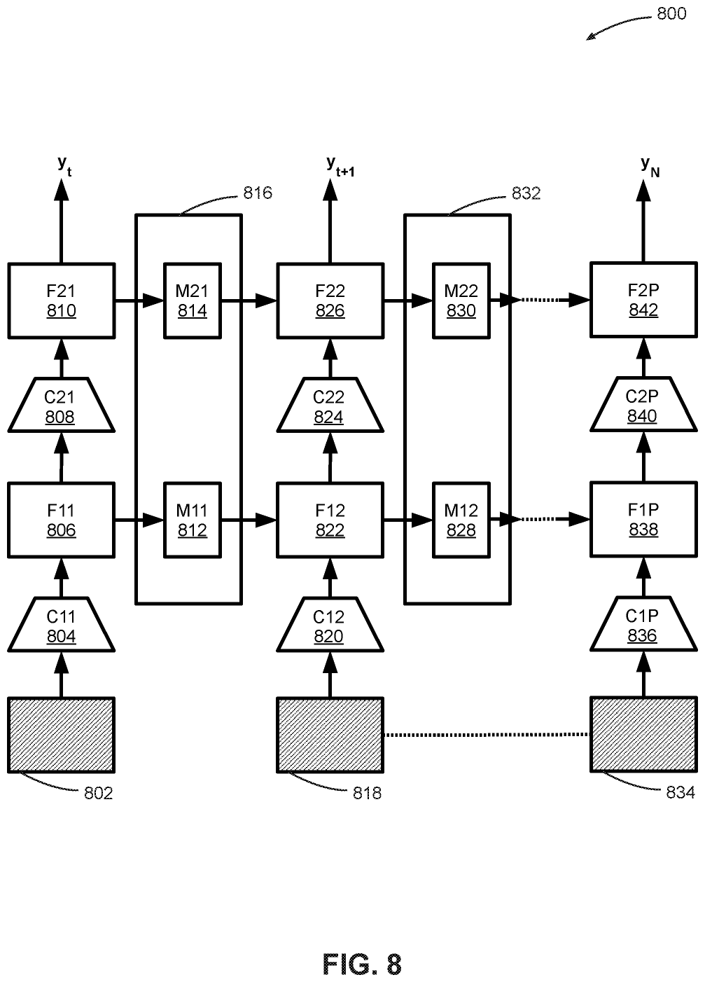

FIG. 8 is a schematic diagram depicting an embodiment of a temporal shift module 800. In some embodiments, temporal shift module 800 is configured to process video frames generated by visual sensor 506. In particular embodiments, temporal shift module 800 is included as a part of a convolutional neural network architecture In some embodiments, visual sensor 506 is an RGB sensor; in other embodiments, visual sensor 506 is a depth sensor. Using a depth sensor instead of an RGB sensor allows a preservation of privacy associated with user 112. In other embodiments visual sensor 506 includes a depth sensor and an RGB sensor and/or other sensors.

In some embodiments, temporal shift module 800 receives a temporal sequence of video frames—a video frame 802 at a time instant t, a video frame 818 at a time instant t+1, through a video frame 834 at a time instant t_{N}. Video frame 802 is processed by a convolutional layer C11 804, which outputs processed data to a feature extractor F11 806. Feature extractor F11 806 is configured to extract one or more features from video frame 802. An output of feature extractor F11 806 is received by a convolutional layer C21 808. The output of convolutional layer C21 808 is received by a feature extractor F21 810 that is configured to extract one or more features from the data processed by convolutional layer C21 808. An output yₜ is generated by feature extractor F21 810, as shown in FIG. 8. In some embodiments, each convolutional layer (e.g., convolutional layer C11 804) is a convolutional block comprised of a plurality of convolutional layers with residual connections. An output of a convolutional block is a feature map of dimensions 1×C×H×W, where C is a number of channels, and (H, W) is a size of a feature map associated with a video frame such as video frame 802.

In some embodiments, an output generated by feature extractor F11 806 and an output generated by feature extractor F21 810 are cached in a memory 816. Specifically, an output of feature extractor F11 806 is shifted out to a memory element M11 812 included in memory 816, and an output of feature extractor F21 810 is shifted out to a memory element M21 814 included in memory 816.

In some embodiments, video frame 818 is received by a convolutional layer C12 820, which outputs processed data to a feature extractor F12 822. Feature extractor F12 822 is configured to extract one or more features from video frame 818. In some embodiments, feature extractor F12 822 receives a replacement input from memory element M11 812. Essentially, the replacement input is a part of a feature from memory element M11 812 that is copied to an output of feature extractor F12 822. An output of feature extractor F12 822 is received by a convolutional layer C22 824. The output of convolutional layer C22 824 is received by a feature extractor F22 826 that is configured to extract one or more features from the data processed by convolutional layer C22 824. In some embodiments, feature extractor F22 826 receives a replacement input from memory element M21 814. An output yₜ₊₁ is generated by feature extractor F22 826.

The above processing flow described to generate the output yₜ₊₁ is similar to the flow described above to generate yₜ, with the difference being that the former processing flow uses additional inputs from memory element M11 812 and memory element M21 814. In some embodiments, the replacement inputs associated with temporal shift module 800 are shifted parts of video channels associated with a stream of video frames along temporal dimension t, t+1, through t_{N}. This process enables a use of temporal information among neighboring video frames which, in turn, enables temporal modeling in an efficient way. In some embodiments, an output generated by feature extractor F12 822 and an output generated by feature extractor F22 826 are cached in a memory 832. Specifically, an output of feature extractor F12 822 is shifted out to a memory element M12 828 included in memory 832, and an output of feature extractor F22 826 is shifted out to a memory element M22 830 included in memory 832.

Video frame 802 at time t through video frame 834 at time N comprise a temporal sequence of video frames, and the two processing flows described above for the video frame 802 and video frame 818 represent a processing flow for each time instant. This process flow is repeated for each time instant, through time instant N, where video frame 834 is received by a convolutional layer C1P 836, which outputs processed data to a feature extractor F1P 838. Feature extractor F1P 838 is configured to extract one or more features from video frame 834. In some embodiments, feature extractor F1P 838 receives a replacement input from a memory element M1(P−1) (not shown but implied in FIG. 8). An output of feature extractor F1P 838 is received by a convolutional layer C2P 840. The output of convolutional layer C2P 840 is received by a feature extractor F2P 842 that is configured to extract one or more features from the data processed by convolutional layer C2P 840. In some embodiments, feature extractor F2P 842 receives a replacement input from a memory element M2(P−1) (not shown but implied in FIG. 8). An output y_{N} is generated by feature extractor F2P 842.

During a training process associated with temporal shift module 800, a video is converted into frames in a time order. In FIG. 8, this time order is shown as being from time instant t through time instant N. In some embodiments, one or more feature channels associated with temporal shift module 800 can be temporally shifted in the future and the past. However, during inference, frames arrive into the system in an online fashion.

At each time instant, a current video frame (such as video frame 802) is processed, and a part of one or more video channels associated with the current video frame are shifted out using data associated with a video channel from a previous time instant. For example, in FIG. 8, feature extractor F12 822 receives such data from memory element M11 812. As shown in FIG. 8, previous channels are cached in memory such as memory 816 and memory 832. In some embodiments, outputs yₜ, yₜ₊₁, through y_{N} are action category predictions by temporal shift module 800, at time instants, t, t+1, through N respectively. In some embodiments, the architecture of temporal shift module 800 as shown in FIG. 8 can be expanded to include additional convolutional layers (e.g., C31, C41, and so on.)

Some embodiments of remote health monitoring system 102 use an RGB sensor as visual sensor 506. In other embodiments, remote health monitoring system 102 uses a depth sensor as visual sensor 506. In an instance when a depth sensor is used, to feed a depth frame into an associated neural network model, the following normalization step may be performed:

Normalize_{d}=d−min(d)/max(d)−min(d)

In some embodiments, machine learning module 302 is subject to an initial training process that uses one or more datasets as a basis for training machine learning module 302. In some embodiments, a public dataset such as Nanyang Technological University's Red Blue Green and Depth information (NTU RGB-D) dataset, with 60 action classes, is used. In other embodiments, a demo room dataset may be generated in a laboratory or a demo room. In an embodiment, a demo room dataset may be comprised of approximately 5000 video clips generated in a demo room.

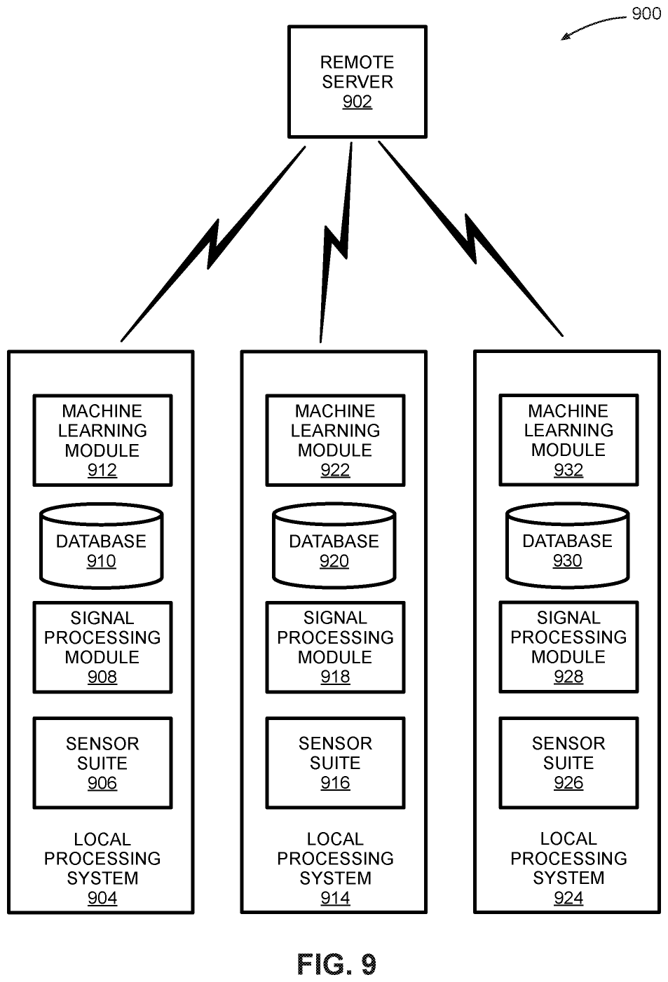

FIG. 9 is a block diagram depicting an embodiment of a remote health monitoring system 900 with privacy-preserving features. In some embodiments, a portion of remote health monitoring system 900 is implemented using a local processing system 904, where local processing system 904 is configured to be in a vicinity of a user such as user 112. Local processing system 904 includes a machine learning module 912 that is configured to implement one or more machine learning algorithms to intelligently identify an activity and detect a condition associated with user, using the algorithms and methods described herein. In some embodiments, machine learning module 912 may be implemented using any combination of a neural network, a support vector machine, a linear regression, and any other machine learning algorithm. In particular embodiments, machine learning module 912 performs functions that are similar to those performed by machine learning module 302.

In some embodiments, local processing system 904 includes a database 910 that is configured to store data associated with user 112. Database 910 may include data associated with one or more activities performed by user 112. Database 910 may also include multiple sets of quantitative data associated with one or more user states, as described herein. A signal processing module 908 included in some embodiments of local processing system 904 performs functions similar to signal processing module 104. A sensor suite 906 is included in some embodiments of local processing system 904. Sensor suite 906 may be comprised of a plurality of sensors such as sensor 1 106 through sensor N 110.

In some embodiments, local processing system 904 is communicatively coupled with a remote server 902 via one or more connectivity methods such as WiFi, Ethernet, a public network, the Internet, and so on. In some embodiments, remote server 902 is instantiated as a cloud-based system.

Some embodiments of remote health monitoring system 900 may include a local processing system 914 and a local processing system 924, where each of local processing system 914 and local processing system 924 functions in a manner similar to local processing system 904. Specifically, local processing system 914 includes a machine learning module 922, a database 920, a signal processing module 918, and a sensor suite 916 that perform similar functions as machine learning module 912, database 910, signal processing module 908, and sensor suite 906 respectively. Similarly, local processing system 924 includes a machine learning module 932, a database 930, a signal processing module 928, and a sensor suite 926 that perform similar functions as machine learning module 912, database 910, signal processing module 908, and sensor suite 906 respectively. Each of local processing system 914 and local processing system 924 is communicatively coupled with remote server 902 as shown in FIG. 9, similar to local processing system 904.

In some embodiments, each of local processing system 904, local processing system 914, and local processing system 924 is associated with a unique user (for example local processing system 904 being associated with a first user, local processing system 914 being associated with a second user, and so on), and each of local processing system 904, local processing system 914, and local processing system 924 performs functions similar to remote health monitoring system 102. In particular embodiments, each of local processing system 904, local processing system 914, and local processing system 924 may be implemented on a separate edge device.

In implementations user (or patient) privacy is an important functional characteristic of remote health monitoring system 102. A specific implementation of a patient privacy preserving remote health monitoring system is depicted in FIG. 9, as remote health monitoring system 900. Specifically, each of local processing system 904 through local processing system 924 does not transmit any data associated with patient privacy to remote server 902. An example of data associated with patient privacy is raw sensor data generated by sensor suite 906, sensor suite 916, and sensor suite 926. This raw sensor data may be directly associated with user bodily functions such as respiration, heartbeat, and so on. Any data processing output from signal processing module 908, signal processing module 918, and/or signal processing module 928 may include data elements that are associated with patient privacy.

In some embodiments, sensor suite 906 may deploy any associated sensors in a distributed architecture. For example, the sensor suite may include a radar located in a vicinity of a user in an environment, while a depth sensor may be located in the environment at a different location relative to the radar.

In some embodiments, signal processing module 908 may be configured with user privacy-preserving features such that no user-identifying information associated with any set of quantitative data (e.g., the first set of quantitative data through the third set of quantitative data) is communicated more than 100 meters to or from signal processing module 908. In particular embodiments, no data processed by signal processing module 908 is communicated more than 100 meters to or from signal processing module 908. In some embodiments, signal processing module 918 and signal processing module 928 include similar privacy-preserving features. In other embodiments, the distance of communication may be less than or greater than 100 meters. For example, a large healthcare/hospital campus may have elements of a local processing system distributed over a large area such that user-identifying information is communicated more than 100 meters to or from signal processing module 908, but no user-identifying information is communicated offsite of the healthcare/hospital campus. In an embodiment implemented at a smaller healthcare facility, such as a small neighborhood care center, the distance of communication could be less than 100 meters—for example with no user-identifying information being communicated more than 50 meters to or from signal processing module 908. As used herein the phrase “healthcare campus” is defined as the grounds, including the buildings, of a healthcare site. A healthcare campus, as that phrase is used herein, may in implementations include only a single building, and in other implementations may include many buildings spanning a city block and more.

Some embodiments of remote health monitoring system 900 implement machine learning algorithms on remote server 902. These machine learning models are configured to process data transmitted by each of local processing system 904 through local processing system 924 to generate personalized models for a user associated with each of local processing system 904 through local processing system 924. In some embodiments, these personalized models are generated independently of any user-identifying data. To achieve this, a federated learning architecture employing machine learning systems such as neural networks are implemented on remote server 902. In some embodiments, each of local processing system 904 through local processing system 924 transmits weighting value updates associated with a neural network at a time instant t. For example, local processing system 904 transmits a weighting value update Δω¹ to remote server 902, local processing system 914 transmits a weighting value update Δω² to remote server 902, and local processing system 924 transmits a weighting value update Δω³ to remote server 902. Remote server 902 then processes these weighting value updates. FIG. 9 shows three local processing systems; for a generalized case with K local processing systems, a neural network associated with the weighting value updates to produce an output:

$${\sum }_{k = 1}^{K} \frac{{n}_{k}}{n} Δ {w}^{k}$$

In the above equation, Δω^{k} is a weight, or weighting parameter, associated with the neural network as discussed above; nₖ is a coefficient associated with each of the K local processing systems (e.g., 904, 914, 924); and n is a sum of all nₖs. In some embodiments, nₖ can be an identical value for each of the K local processing systems, or nₖ can be determined based on heuristics such as a frequency of usage associated with the K local processing systems. The output generated in the above equation is fed back as an update to each of the K local processing systems. This update/feedback loop cycle continues in an ongoing manner, where training and updating the neural network model is accomplished without accessing any user data. This process preserves user/patient privacy by preventing any transmission of user-sensitive data over a public network.

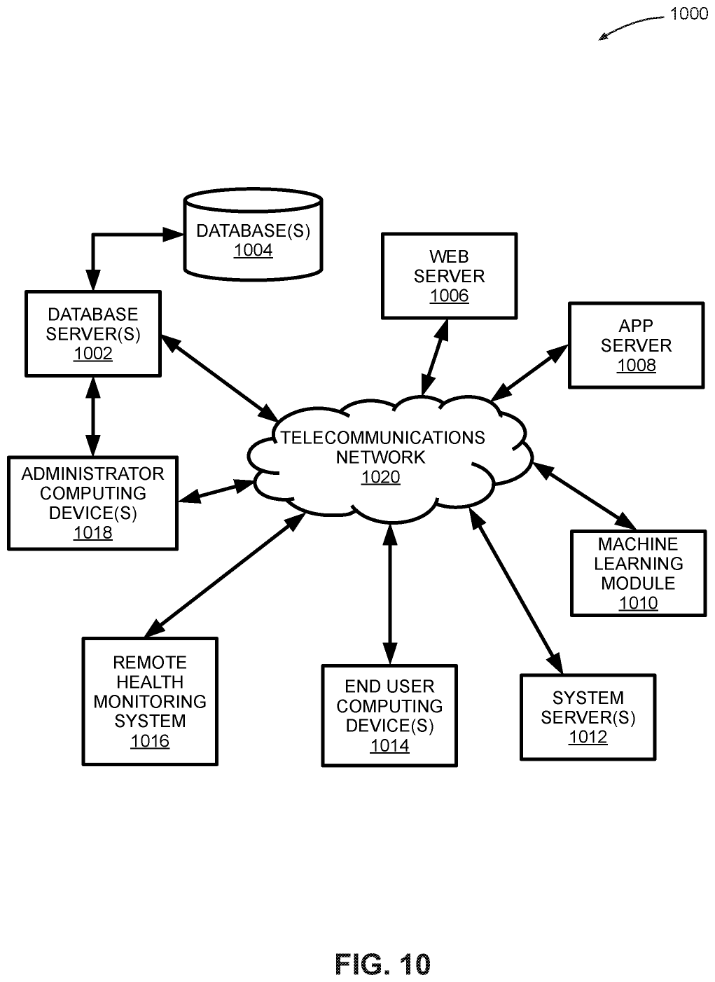

FIG. 10 is a block diagram depicting an embodiment of a system architecture 1000 of a remote health monitoring system. In some embodiments, architecture 1000 includes a remote health monitoring system 1016 that includes the functionalities, subsystems and methods described herein. Remote health monitoring system is coupled to a telecommunications network 1020 that can include a public network (e.g., the Internet), a local area network (LAN) (wired and/or wireless), a cellular network, a WiFi network, and/or some other telecommunication network.

Remote health monitoring system 1016 is configured to interface with an end user computing device(s) 1014 via telecommunications network 1020. In some embodiments, end user computing device(s) can be any combination of computing devices such as desktop computers, laptop computers, mobile phones, tablets, and so on. For example, an alarm generated by remote health monitoring system 1016 may be transmitted through the telecommunications network to an end user computing device in a hospital to alert associated medical personnel of an emergency (e.g., a fall).

In some embodiments, remote health monitoring system 1016 is configured to communicate with a system server(s) 1012 via telecommunications network 1020. System server(s) 1012 is configured to facilitate operations associated with system architecture 1000, for example signal processing modules 104, 908, 918, 928 may be implemented using one or more servers communicatively coupled with sensors.

In some embodiments, remote health monitoring system 1016 communicates with a machine learning module 1010 via telecommunications network 1020. Machine learning module 1010 is configured to implement one or more of the machine learning algorithms described herein, to augment a computing capability associated with remote health monitoring system 1016. Machine learning module 1010 could be located on one or more of the system server(s) 1012.

In some embodiments, remote health monitoring system 1016 is enabled to communicate with an app server 1008 via telecommunications network 1020. App server 1008 is configured to host and run one or more mobile applications associated with remote health monitoring system 1016.

In some embodiments, remote health monitoring system 1016 is configured to communicate with a web server 1006 via telecommunications network 1020. Web server 1006 is configured to host one or more web pages that may be accessed by remote health monitoring system 1016 or any other components associated with system architecture 1000. In particular embodiments, web server 1006 may be configured to serve web pages in a form of user manuals or user guides if requested by remote health monitoring system 1016, may allow administrators to monitor operation and/or data collection of the remote health monitoring system 100, adjust system settings, and so forth remotely or locally.

In some embodiments a database server(s) 1002 coupled to a database(s) 1004 is configured to read and write data to database(s) 1004. This data may include, for example, data associated with user 112 as generated by remote health monitoring system 102.

In some embodiments, an administrator computing device(s) 1018 is coupled with telecommunications network 1020 and with database server(s) 1002. Administrator computing devices(s) 1018 in implementations is configured to monitor and manage database server(s) 1002, and monitor and manage database 1004 via database server(s) 1002. It may also allow an administrator to monitor operation and/or data collection of the remote health monitoring system implementation 100, adjust system settings, and so forth remotely or locally.

Although the present disclosure is described in terms of certain example embodiments, other embodiments will be apparent to those of ordinary skill in the art, given the benefit of this disclosure, including embodiments that do not provide all of the benefits and features set forth herein, which are also within the scope of this disclosure. It is to be understood that other embodiments may be utilized, without departing from the scope of the present disclosure.

What is claimed is:

1. An apparatus configured to perform local processing of one or more user states associated with a user, the apparatus comprising:
a plurality of sensors configured for contact-free monitoring of at least one user state; and
a signal processing module communicatively coupled with the plurality of sensors, wherein the signal processing module is configured to receive data from the plurality of sensors;
wherein a first sensor of the plurality of sensors is configured to generate a first set of quantitative data associated with a first user state;
wherein a second sensor of the plurality of sensors is configured to generate a second set of quantitative data associated with a second user state;
wherein a third sensor of the plurality of sensors is configured to generate a third set of quantitative data associated with a third user state;
wherein the signal processing module is configured to process the first set of quantitative data, the second set of quantitative data, and the third set of quantitative data, using a machine learning module,
wherein the signal processing module is configured to, responsive to the processing, one of identify a user activity and detect a condition associated with the user; and
wherein no user-identifying information of the first through third sets of quantitative data and no user-identifying information of the processed data is communicated more than 100 meters from or to the signal processing module.

2. The apparatus of claim 1, wherein the user activity includes one of sitting, standing, walking, sleeping, eating, undressing, dressing, washing face, washing hands, brushing teeth, brushing hair, using a toilet, putting on dentures, removing dentures, and laying down.

3. The apparatus of claim 1, wherein the condition is one of a fall, a health condition, and a triage severity.

4. The apparatus of claim 1, wherein the signal processing module is configured to generate an alarm in response to detecting a condition that is detrimental to the user.

5. The apparatus of claim 1, wherein the signal processing module and the plurality of sensors are configured in a hub architecture wherein the plurality of sensors are removably coupled with the signal processing module.

6. The apparatus of claim 1, wherein the signal processing module includes one of a GPU, a CPU, an FPGA, and an AI computing chip.

7. The apparatus of claim 1, wherein the plurality of sensors includes one of a depth sensor, an RGB sensor, a thermal sensor, a radar sensor, and a motion sensor.

8. The apparatus of claim 1, wherein the signal processing module characterizes the user activity using a convolutional neural network.

9. The apparatus of claim 8, wherein the convolutional neural network includes a temporal shift module.

10. The apparatus of claim 1, wherein the signal processing module is implemented using an edge device.

11. A method to perform contact-free monitoring of one or more user activities, the method comprising:
generating, using a first sensor of a plurality of sensors, a first set of quantitative data associated with a first user state of a user, wherein the first sensor does not contact the user;
generating, using a second sensor of the plurality of sensors, a second set of quantitative data associated with a second user state, wherein the second sensor does not contact the user;
generating, using a third sensor of the plurality of sensors, a third set of quantitative data associated with a third user state, wherein the third sensor does not contact the user;
processing, using a signal processing module and using a machine learning module, the first set of quantitative data, the second set of quantitative data, and the third set of quantitative data, wherein the signal processing module is communicatively coupled with the plurality of sensors;
responsive to the processing, identifying, using the signal processing module, one or more user activities; and
responsive to the processing, detecting, using the signal processing module, a condition associated with the user;
wherein the plurality of sensors and the signal processing module are located at a healthcare campus, and wherein no user-identifying information of the first through third sets of quantitative data and no user-identifying information of the processed data is communicated offsite of the healthcare campus.

12. The method of claim 11, wherein the one or more user activities includes one of sitting, standing, walking, sleeping, eating, undressing, dressing, washing face, washing hands, brushing teeth, brushing hair, using a toilet, putting on dentures, removing dentures, and laying down.

13. The method of claim 11, wherein the condition is one of a fall, a health condition, and a triage severity.

14. The method of claim 11, further comprising generating an alarm, using the signal processing module, in response to detecting a condition that is detrimental to the user.

15. The method of claim 11, wherein the signal processing module and the plurality of sensors are configured in a hub architecture wherein the plurality of sensors are removably coupled with the signal processing module.

16. The method of claim 11, wherein the signal processing module includes one of a GPU, a CPU, an FPGA, and an AI computing chip.

17. The method of claim 11, wherein the plurality of sensors includes a thermal sensor, a radar sensor, and one of a depth sensor and an RGB sensor.

18. The method of claim 11, further comprising characterizing one or more user activities using a convolutional neural network associated with the signal processing module.

19. The method of claim 18, wherein the convolutional neural network includes a temporal shift module.

20. The method of claim 11, wherein the signal processing module comprises an edge device.
